SEQUENCE LISTING (5 sequences)

SEQ ID NO: 1 - protein, 404 aa, Homo sapiens
  misc_feature: CD209_HUMAN
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 2 - protein, 1333 aa, Severe acute respiratory syndrome coronavirus 2
  misc_feature: SPIKE_SARS2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 399 aa, Homo sapiens
  misc_feature: CLC4M_HUMAN
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 4 - protein, 1315 aa, Severe acute respiratory syndrome coronavirus
  misc_feature: SPIKE_CVHSA
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 5 - protein, 547 aa, Homo sapiens
  misc_feature: Human Intercellular adhesion molecule 3
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

(19) United States
(12) Patent Application Publication
(10) Pub. No.: US 2023/0172950 A1
(43) Pub. Date: Jun. 8, 2023

(54) Glucocorticoid Receptor (GR) Modulators for Treating a SARS-COV-2 Virus

(71) Applicant: AVM Biotechnology, LLC, Seattle, WA (US)

(72) Inventors: Theresa Deisher, Seattle, WA (US); Scot Wayne McKay, Seattle, WA (US)

(73) Assignee: AVM Biotechnology, LLC, Seattle, WA (US)

(21) Appl. No.: 17/921,128

(22) PCT Filed: Apr. 28, 2021
(86) PCT No.: PCT/US2021/029608

§ 371 Date: Oct. 25, 2022

Related U.S. Application Data
(60) Provisional application No. 63/017,199, filed on Apr. 29, 2020.

(51) Int. Cl. A61K 31/573; A61P 31/14

(52) U.S. Cl. CPC A61K 31/573; A61P 31/14

(57) ABSTRACT

The present disclosure relates to novel therapies and associated methods of treatment based on newly provided therapeutic mechanisms mediated by the binding of glucocorticoids to intercellular adhesion molecules (ICAMs).

### FIELD OF THE INVENTION

The present invention provides novel therapies based on newly provided therapeutic mechanisms. More particularly, the inventors have found that glucocorticoids can act through a previously unknown mechanism, involving binding to intercellular adhesion molecules (ICAMs). This opens up new therapeutic modalities for treating life threatening diseases such as the severe acute respiratory condition COVID-19, caused by SARS-COV-2.

### BACKGROUND

Adhesion molecules are glycoproteins expressed on cell surfaces, which mediate the contact between two cells (both homotypic and heterotypic interactions) or between cells and the extracellular matrix (Hua et al, 2013, which is hereby incorporated by reference in its entirety). ICAMs are type I transmembrane glycoproteins of the immunoglobulin superfamily, which are ligands for antigens expressed on the surface of immune cells, such as leukocyte integrins.

ICAM-1 is known to be the major surface receptor for rhinoviruses (Staunton et al, 1989; Bhella, 2015; both of which are hereby incorporated by reference in their entirety). While ICAM-1 has been the primary focus of much research (Hua et al), there are four other members of the ICAM family, denoted ICAM2, 3, 4 and 5.

ICAM3 (also known as CD50) is expressed by lymphocytes, monocytes, eosinophils and neutrophils (as well as by lymphoma cells and some melanoma, sarcoma and other cancer cells, as well as bronchiole epithelial cells). Information on the underlying ICAM3 gene is available online, for instance on the Ensemble database; see entry ENSG00000076662. ICAM3-mediated signaling proceeds via recruitment of Src by the YLPL motif in the ICAM3 intracellular domain leading to PI3K-AKT phosphorylation cascades (Shen et al, 2018, which is hereby incorporated by reference in its entirety). ICAM3 expression on eosinophils is decreased by exposure to modest concentrations of dexamethasone (100 pM to 1 μM) (Juan et al, 1999, which is hereby incorporated by reference in its entirety). ICAM3 is a candidate cell-entry receptor for viruses. For instance, ICAM3 has been proposed to play a role in HIV-1 entry, since some antibodies specific for ICAM3 significantly inhibit the early events in the virus life cycle (Sommerfelt and Asjo, 1995; Barat et al, 2004; both of which are hereby incorporated by reference in their entirety).

As discussed in more detail below, certain ICAM3 gene variants are reportedly associated with the pathogenesis of severe acute respiratory syndrome (SARS) (Chan et al, 2007, which is hereby incorporated by reference in its entirety).

ICAM4 was originally named the ‘LW glycoprotein’ and its expression was thought to be largely restricted to red blood cells, although more recent studies have shown it to be expressed also by macrophages (Choi et al, 2017, which is hereby incorporated by reference in its entirety). Information on the underlying ICAM4 gene is available online, for instance on the Ensemble database; see entry ENSG00000105371. ICAM4 is a candidate cell-entry receptor for some pathogens, such as Mycobacterium tuberculosis and Plasmodium falciparum (Bhalla et al, 2015).

Coronavirus is a family of single stranded RNA viruses. There are four types of coronaviruses; a coronavirus (α-COV), β coronavirus (β-COV), γ coronavirus (γ-COV) and δ coronavirus (δ-COV) (Chan et al, 2013, which is hereby incorporated by reference in its entirety). Several coronaviruses are known to cause disease in humans. Following deadly SARS and MERS epidemics caused by p coronaviruses SARS-CoV (also referred to as SARS-Cov-1 or ‘CoV1’ herein) and MERS-CoV respectively, another p coronavirus resulted in a major pandemic disease referred to as COVID-19, which spread across the world in 2020. The cause of COVID-19 is the SARS-CoV-2 virus, which is approximately 79% homologous to the original SARS-CoV by genome sequence homology (Wang et al, 2020, which is hereby incorporated by reference in its entirety) and is even more closely related to a SARS-like coronavirus (MG772933) that infects bats (Wu et al, 2020, which is hereby incorporated by reference in its entirety).

Coronaviruses were thought to mainly recognize the corresponding receptor on the target cell through the spike (S) glycoprotein on its surface and enters into the cell, then causing the infection (Wang et al, 2020). Some analysis indicated that SARS-CoV-2 uses angiotensin-converting enzyme 2 (ACE2) as its cell-entry receptor, as did SARS-CoV-1; and structure-model analysis showed that SARS-CoV-2 binds ACE2 with an affinity more than 10 fold higher than does SARS-CoV, higher than the threshold required for virus infection (Wrapp et al, 2020). However, the detailed mechanism on whether SARS-CoV-2 infects humans via binding of S-protein to ACE2, how strong the interaction is to effect human transmission, and how SARS-CoV-2 causes pathological disease and organ damage remains unknown, and needs more studies to elaborate (Wang et al, 2020). As of April 2020, no vaccine or specific antiviral treatments existed for COVID-19, with management of the disease focused on treatment of symptoms and supportive care.

Studies into the SARS outbreak caused by SARS-CoV-1 found that, besides binding to angiotensin-converting enzyme-2 (ACE2), additionally, the C-type lectins L-SIGN (liver-specific ICAM3 grabbing nonintegrin) and DC-SIGN (dendritic cell-specific ICAM3 grabbing nonintegrin) were reported to constitute “second” or “supporting” receptors, which also play a role in facilitating viral cell entry (Jia et al, 2005; Kuba et al, 2006; both of which are hereby incorporated by reference in their entirety). It was later reported that L-SIGN and DC-SIGN are SARS-CoV-1 receptors that are independent of ACE2, but that N-linked glycosylation at several asparagine residues of the SARS-CoV-1 S glycoprotein is critical for L-SIGN/DC-SIGN mediated cell entry (Han et al, 2017, which is hereby incorporated by reference in its entirety). L-SIGN is present in the liver, lymph node and lung (Han et al, 2017).

N-glycosylation is also important for ICAM3 function. ICAM3 is the most heavily glycosylated protein in the ICAM family, having five N-linked glycosylation sites on the ligand-binding domain (Song et al, 2005, which is hereby incorporated by reference in its entirety).

Early studies assume that SARS-Cov-2 primarily enters human cells via ACE2. However, it has been reported that lung alveolar type II (AT2) cells, which were believed to be the main target of SARS-Cov-2, express low levels of ACE2; several other possible receptors for SARS-Cov-2 entry to AT2 cells are under investigation (Qi et al, 2020, which is hereby incorporated by reference in its entirety).

The present authors have previously found that high concentrations of glucocorticoids could be used to cause lymphodepletion in patients, e.g. to treat lymphocyte mediated diseases (as described in WO2020/072713A1, which is hereby incorporated by reference in its entirety) or to enhance the efficacy of cellular immunotherapies such as adoptive T cell therapy (described in WO2018/183927, which is hereby incorporated by reference in its entirety). However, to date it has not been suggested that high dose glucocorticoids could act at viral entry points by blocking viral cell-entry receptors.

A need exists for further treatments for viral infections such as COVID-19. Treatments that are simple and associated with low toxicity and cost are especially desired.

The present invention has been devised in light of the above considerations.

### SUMMARY OF THE INVENTION

The present invention is based on the finding that, following high dose administration, glucocorticoid molecules can bind and block intercellular adhesion molecules such as ICAM3. The binding is cooperative and up to 26 molecules bind the first Ig domain of ICAM3. The inventors also identify substantial functional homology between the spike (S) glycoprotein of SARS-Cov-2 and DC-SIGN and L-SIGN, indicating that, like DC-SIGN and L-SIGN, the SARS-Cov-2 S glycoprotein should bind ICAM3, as well as potentially binding L-SIGN and DC-SIGN. The inventors then performed molecular modelling studies and found the binding energy between the receptor binding domain (RBD) of SARS-Cov-2 and ICAM3 to be more favorable than the binding energy between the RBD of SARS-Cov-2 and ACE2. Similarly, preliminary MicroScale Thermophoresis experiments carried out by the inventors indicate that the binding affinity (KD) of the receptor binding domain (RBD) of SARS-Cov-2 and ICAM3 is lower than that for the RBD of SARS-Cov-2 and ACE2. These findings led the inventors to the striking conclusion that high dose glucocorticoid therapy could have multiple, cooperative actions that should inhibit SARS-Cov-2 from infecting target cells. This represents an important new approach to treating COVID-19.

When administered at high doses, the glucocorticoids can be ‘soaked up’ by ICAM3, which is expressed at substantial levels on cells such as lymphocytes, monocytes and neutrophils and also expressed on bronchiole cells, and thus not act via glucocorticoid receptors. Without being bound by theory, the inventors believe this binding to ICAM3 can lead to the production and mobilization of novel Natural Killer T (NKT) cells from the spleen, thymus, or bone marrow into the circulation. These novel NKT cells can identify and target SAR CoV2 infected cells for destruction. Glucocorticoid binding to ICAM3 may also lead to the cell becoming marked for attack by lymphocytes such as NKT cells and CD8+ T cells. ICAM3 shedding may also occur following glucocorticoid binding, further stimulating an immune response as well as preventing the SARS-CoV-2 virus from entering the body via the respiratory tract if ICAM3 is the primary receptor for cell entry. ICAM3 is one of the most heavily Asp-glycosylated proteins in the human body, which could contribute to its action in blocking SARS-CoV-2 cell entry e.g. once solubilized. Additionally, the binding of glucocorticoid molecules such as dexamethasone to ICAM3 may enhance its binding to L-SIGN and DC-SIGN, thereby displacing SARS-CoV-2 from binding these proteins.

Moreover, as described above, the inventors consider ICAM3 to represent an important point of entry for the SARS-CoV-2 virus to enter cells; particularly immune cells, which highly express ICAM3. Thus, glucocorticoid occupancy of ICAM3 inhibits SARS-CoV-2 from entering these cells, providing an important way of protecting the patient's immune system from being compromised by the virus. Furthermore, if the cell does become infected with SARS-Cov-2, glucocorticoid occupancy of ICAM3 marks the infected cell for destruction e.g. via the mechanisms described above. Thus, ICAM3 binding by this class of agents represents a potentially synergistic way of curbing the spread of SARS-CoV-2 virus within an individual. It follows that the treatments described herein represent a potentially important way of curbing the spread of COVID-19.

Accordingly, in a first aspect, this invention provides a method of inhibiting a SARS-Cov-2 virus from entering a host cell, the method comprising contacting the cell with a glucocorticoid receptor (GR) modulating agent. The GR modulating agent may act by binding to Intercellular Adhesion Molecule 3 (ICAM3) present at the surface of the host cell, e.g. as an ICAM3 agonist or antagonist. Thus, the GR modulating agent can inhibit the spike (S) glycoprotein of SARS-Cov-2 from binding to the ICAM3 present at the surface of the host cell. In some embodiments, the GR modulating agent causes ICAM3 shedding from the surface of the host cell into the extracellular space. In the respiratory tract this ICAM3 shedding may prevent entry of the SARS-CoV-2 virus into the body. In some embodiments, the GR modulating agent contacts an additional cell and causes ICAM3 shedding from the surface of the additional cell into the extracellular space. The ICAM3 that is shed into the extracellular space may inhibit SARS-Cov-2 from binding to the surface of the host cell. In some embodiments, ICAM3 that has been shed into the extracellular space inhibits SARS-Cov-2 binding to L-SIGN and/or DC-SIGN at the surface of the host cell. In some embodiments, ICAM3 that has been shed into the extracellular space binds to SARS-Cov-2 itself, thereby reducing its binding to ICAM3, L-SIGN and/or DC-SIGN at the surface of the host cell.

In some embodiments, the GR modulating agent acts by binding SARS-Cov-2 to block its binding to its viral entry receptor on the host cell.

In some embodiments, the host cell is an immune cell, e.g. a lymphocyte, a monocyte, an eosinophil, a neutrophil or a dendritic cell. In some embodiments, the host cell is a lung cell, e.g. an alveolar type-2 cell or a bronchiole cell, such as a bronchiole epithelial cell.

As discussed herein, the GR modulating agent may be a glucocorticoid. In some embodiments, the glucocorticoid is selected from the group consisting of: dexamethasone, hydrocortisone, methylprednisolone, prednisone, prednisolone, prednylidene, cortisone, budesonide, betamethasone, flumethasone and beclomethasone. Preferably, the glucocorticoid is dexamethasone, e.g. dexamethasone base or dexamethasone sodium phosphate.

In a further aspect, the invention provides a method of treating COVID-19 in a patient, the method comprising administering a glucocorticoid receptor (GR) modulating agent at a sufficiently high dose to inhibit SARS-Cov-2 particles from infecting a cell of the patient and/or at a sufficiently high dose to support or trigger an effective immune response to SARS-Cov-2 in the patient. In some embodiments, the GR modulating agent may have a direct killing effect on ICAM3 expressing cells, which may be infected with SARS-Cov-2.

In a related aspect, the invention provides a glucocorticoid receptor (GR) modulating agent for use in a method of treating COVID-19 in a patient, the method comprising administering the GR modulating agent at a sufficiently high dose to inhibit SARS-Cov-2 particles from infecting a cell of the patient and/or at a sufficiently high dose to support or trigger an effective immune response to SARS-Cov-2 in the patient.

In a related aspect, the invention provides the use of a glucocorticoid receptor (GR) modulating agent in the manufacture of a medicament for treating COVID-19 in a patient, wherein the treatment comprises administering the medicament at a sufficiently high dose to inhibit SARS-Cov-2 particles from infecting a cell of the patient and/or to support or trigger an effective immune response to SARS-Cov-2 in the patient.

In some embodiments, the GR modulating agent is a glucocorticoid, e.g. dexamethasone, hydrocortisone, methylprednisolone, prednisone, prednisolone, prednylidene, cortisone, budesonide, betamethasone, flumethasone and beclomethasone. Preferably, the glucocorticoid is dexamethasone compound or formulation disclosed herein. The dose of the GR modulating agent is at least about 12 mg/kg, at least about 15 mg/kg, at least about 18 mg/kg, at least about 24 mg/kg, at least about 30 mg/kg, or at least about 45 mg/kg human equivalent dose (HED) of dexamethasone base. The patient is preferably a human patient. Thus, where the treatment related aspects of the invention are performed on a human patients, and where a dexamethasone compound is used for the treatment, the dexamethasone dose may be at least about 12 mg/kg, at least about 15 mg/kg, at least about 18 mg/kg, at least about 24 mg/kg, at least about 30 mg/kg, or at least about 45 mg/kg.

In some embodiments, SARS-Cov-2 particles are inhibited from infecting an immune cell of the patient by binding to Intercellular Adhesion Molecule 3 (ICAM3) present at the surface of the immune cell. The GR modulating agent may act as an ICAM3 agonist or antagonist. The GR modulating agent may act to inhibit the spike (S) glycoprotein of SARS-Cov-2 from binding to the ICAM3 present at the surface of the immune cell. The immune cell may be a lymphocyte, a monocyte, an eosinophil, a neutrophil or a dendritic cell.

In some embodiments, SARS-Cov-2 particles are inhibited from infecting a lung cell of the patient. The high dose GR modulating agent may cause ICAM3 shedding (from lung cells and/or other cells) into the extracellular space and this ICAM3, which has been shed into the extracellular space may inhibit SARS-Cov-2 from binding to the surface of the lung cell. The ICAM3 that has been shed into the extracellular space may inhibit SARS-Cov-2 binding to L-SIGN and/or DC-SIGN at the surface of the lung cell. In some embodiments, the lung cell is an alveolar type-2 cell and/or a bronchiole cell. In some embodiments, the lung cell is an alveolar type-2 cell. In some embodiments, the lung cell is a bronchiole cell such as a bronchiole epithelial cell.

In some embodiments, the GR modulating agent acts by binding a SARS-Cov-2 particle, which is thus inhibited from binding to its viral entry receptor on the cell of the patient.

In some embodiments, the treatment of COVID-19 in a patient triggers or supports an effective immune response. (The terminology ‘triggers or supports’ is intended to cover treatments that both trigger and support these immune response; thus the term ‘triggers or supports’ may be substituted for ‘triggers and/or supports’). The effective immune response may be triggered or supported because the high-dose GR modulating agent can induce and/or mobilise immune cells to fight the virus:

The high-dose GR modulating agent may trigger or support an effective immune response by inducing and/or mobilising of a population of NKT cells that are characterized in that they expresses CD3, and:
a. express CD4, CD8, CD45, CD49b (CD56 in humans), CD62L, NK1.1, Ly6G, Sca1, and/or TCR gamma/delta; and/or
b. do not express: C-kit, B220, FoxP3, and/or TCR alpha/beta.

The high-dose GR modulating agent may trigger or support an effective immune response by inducing and/or mobilising a population of T cells that express CD3 to a very high level (“CD3-very-high”).

The high-dose GR modulating agent may trigger or support an effective immune response by inducing and/or mobilising a population of dendritic cells (DCs) that express CD11b to a very high level (“CD11b-very-high dendritic cells”).

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Sequence alignment of human DC-SIGN, L-SIGN, SARS-Cov and SARS-Cov-2 spike glycoproteins. The regions of homology between the SARS-Cov-2 spike and DC/L-SIGN are indicated. This homology is not shared by the SARS-Cov spike glycoprotein.

FIG. 2. Amino acid sequence of ICAM3. The underlined residues are N-linked glycosylated asparagine residues at positions 52, 84, 87, 101, 110, 134, 206, 264, 295, 308, 320, 363, 389, 453 and 457.

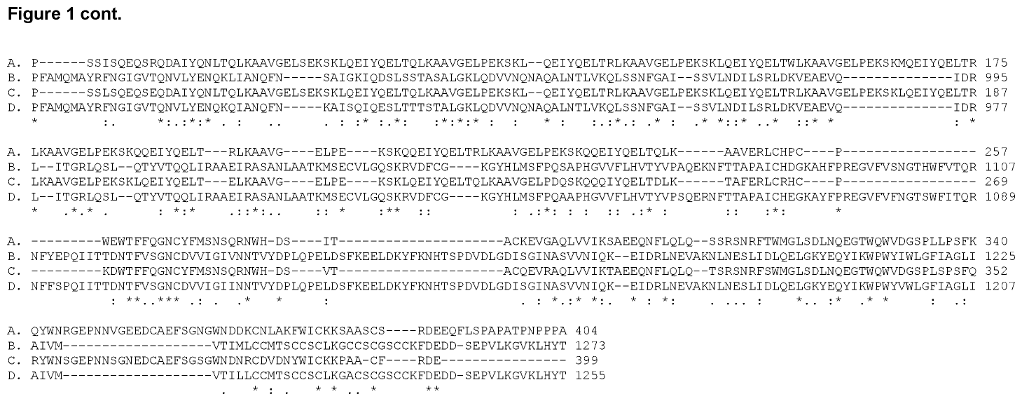

FIG. 3. Splenocyte viability following 4 hours incubation with dexamethasone base (1 nM-250 μM).

FIG. 4. ClusPro model of ACE2 docking with the receptor-binging domain (RBD) of the spike protein of SARS-CoV-2 (left-hand panel). Rosetta Interference Score plot of the docking models (right-hand panel).

FIG. 5. ClusPro model of ICAM3 docking with the receptor-binging domain (RBD) of the spike protein of SARS-CoV-2 (left-hand panel). Rosetta Interference Score plot of the docking models (right-hand panel).

FIG. 6. ClusPro model of ICAM3 docking with the receptor-binging domain (RBD) of the spike protein of SARS-CoV-1 (left-hand panel). Rosetta Interference Score plot of the docking models (right-hand panel).

FIG. 7. Results from an independent repeat of ClusPro modelling and predicted energetics of binding.

FIG. 7A shows the Rosetta Interference Score plot for the repeat docking model of ACE2 docking with the receptor-binging domain (RBD) of the spike protein of SARS-CoV-2. FIG. 7B shows the Rosetta Interference Score plot for the repeat docking model of ICAM3 docking with the receptor-binging domain (RBD) of the spike protein of SARS-CoV-2. FIG. 7C shows the Rosetta Interference Score plot for the repeat docking model of ICAM3 docking with the receptor-binging domain (RBD) of the spike protein of SARS-CoV-1.

FIG. 8. ClusPro model of binding interaction of the SARS-CoV-2 receptor binding domain, ICAM3, and DC-SIGN. When the SARS CoV2 SPIKE RBD (bottom left molecule) binds to ICAM3 (top molecule), then DC-SIGN (bottom right molecule) cannot bind and vice versa.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

SARS CoV2

SARS CoV2 (CoV2) early symptoms are distinct from Influenza-symptoms that lead to Acute Respiratory Distress Syndrome (ARDS), the hallmark of SARS CoV (CoV1). CoV2 early symptoms include bronchiole mediated silent hypoxemia, rather than alveolar driven ARDS. CoV1 SPIKE used ACE2, L-SIGN (sinusoidal endothelial cells) and DC-SIGN (dendritic cells) for infection/pathogenesis. SIGNS bind CoV1 SPIKE outside of the RBD (Jeffers, 2004; Marzi, 2004). Lymphocyte infection by CoV1 was uncommon (Panesar 2008), while lymphocyte (Shih 2020; Feng 2020) and monocyte (Zhang 2020) infection by CoV2 is commonly observed. CoV2 SPIKE but not CoV1 SPIKE can infect T-lymphocytes that express none or little ACE2 (Wang 2020). These data suggest that CoV2 may use other receptors for cell entry. ICAM3 is the most heavily mannosylated (critical for virus binding); its expression is limited to lymphocytes, monocytes, granulocytes and alveolar and bronchiole epithelial cells (Human Proteome Project; ATTC) and has been associated with ARDS severity (Chan 2007), suggesting it as a candidate for CoV2 SPIKE binding. HEK293 cells, common screens for COV2 interactions, do not express ICAM3.

Based on the symptoms of COVID-19 patients and the biology being discovered, which is a disease quite different than SARS CoV1, the inventors have looked for potential receptors for SARS CoV2 entry in addition to hACE2. COVID-19 patients exhibit silent hypoxemia as early symptoms, which is a bronchiole mediated pathology, rather than ARDS which is alveolar mediated. Bronchioles express ICAM3, not ACE2. Additionally, unlike SARS CoV1 which infected dendritic cells via binding to DC-SIGN and L-SIGN, SARS CoV2 infects lymphocytes, monocytes and neutrophils which express ICAM3 heavily and selectively. DC-SIGN and L-SIGN are natural ligands for ICAM3, a receptor selectively expressed on lymphocytes, monocytes, neutrophils, bronchioles and cancer cells (Human Proteome Project).

Reports also suggest that patients using inhaled steroids have 80% protection against infection, while people using ACE inhibitors or receptor blockers have no protection. Inhaled dexamethasone should be expected to cleave ICAM3—in vitro, dexamethasone cleaves ICAM3 from cells, and dexamethasone has been reported to reduce mortality in severely ill COVID-19 patients (Horby et al, 2021).

N-Linked Glycosylation

ICAM3 is one of the most heavily Asp-glycosylated protein in the human body. Asparagine residues (Asp) become decorated with glycans linked at the nitrogen of the amide side group. This is a biologically important posttranslational modification that begins in the endoplasmic reticulum with the transfer of a core glycan comprising three mannose subunits. Further modifications can occur in the Golgi apparatus and/or at the plasma membrane where glycoproteins may be secreted.

Low levels of mannose-binding lectin is prevalent in certain ethnic groups such as Caucasian populations (Siljan et al, 2018, which is hereby incorporated by reference in its entirety). This may associate with a decreased susceptibility to developing severe symptoms or complications arising from certain viral diseases, such COVID-19.

Pharmacological Action

A receptor antagonist is a ligand that binds to the receptor, thus occupying the receptor binding site, but without activating the receptor. In contrast, a receptor agonist binds and activates the receptor, which typically causes a signal to be transduced within the cell. Therefore, antagonists can exert biologically relevant actions by competing with agonists, and other receptor-binding agents, for binding. Antagonists are therefore commonly referred to as ‘blockers’.

The glucocorticoid modulating agents of the present disclosure typically act as agonists on glucocorticoid receptors. However, the present application discloses the surprising capacity of glucocorticoid receptor modulating agents (such as dexamethasone and other glucocorticoids) to bind the Intracellular Adhesion Molecule 3 (ICAM3). Without being bound by theory, the present authors believe that glucocorticoid receptor modulating agents may bind ICAM3 and exert an antagonistic action upon ICAM3 without triggered the normal signalling cascades that are caused by ICAM3 activation.

Other Mechanisms

High doses of glucocorticoid exert powerful biological effects that will contribute to the treatment of COVID-19.

In addition to the mechanisms outlined above, the present authors believe that binding of high doses of glucocorticoid receptor modulating agents to ICAM3 may allow the glucocorticoid receptor modulating agents to exert a concentration dependent, direct killing effect on ICAM3-expressing cells, which may be ICAM3 expressing cells that are infected with SARS-Cov-2.

Thus, in some embodiments, the GR modulating agents may induce cell death of ICAM3 expressing cells following binding to ICAM3. In some embodiments, the GR modulating agents induce apoptosis of ICAM3 expressing cells by binding to ICAM3. In some embodiments, the GR modulating agent triggers or supports an effective immune response against the ICAM3 expressing cells. In some embodiments, the GR modulating agent causes ICAM3 shedding from the surface of a cell into the extracellular space. In some embodiments, the GR modulating agent causes ICAM3 expressing cells to be marked for attack by immune cells. In some embodiments the GR modulating agent directly triggers (activates) cell apoptotic pathways.

Biological Action by Marking Cells for Immune Attack

Monocytes and macrophages are phagocytic white blood cells which act in both non-specific and specific defense mechanisms in vertebrates. Their role is to phagocytose (engulf and then digest) dead and dying cells, cellular debris and pathogens either as stationary or as mobile cells, and to stimulate lymphocytes and other immune cells to respond to the pathogen. The molecular mechanisms underlying recruitment of phagocytic white blood cells is believed to involve recognition of molecular ‘flags’ on the surface of dying cell, that are bound and allow the dying cell to be eaten and destroyed (Gregory & Pound, 2010, hereby incorporated by reference in its entirety). ICAM3 has been shown to act as such a molecular ‘flag’ following a change of function on dying cells (Moffat et el 1999, hereby incorporated by reference in its entirety).

Without being bound by theory, the present authors believe that the direct killing effect of acute high dose glucocorticoids on ICAM3 expressing cells may be mediated by binding of glucocorticoids to ICAM3 causing the ICAM3 expressing cells to become marked for attack by macrophages and/or leukocytes such as NKT cells and CD8+ T cells. These may include the immune cells that the present authors have shown are induced/mobilised by high concentrations of glucocorticoids, as described more fully below.

ICAM3 shedding following glucocorticoid binding may further stimulate an immune response against these cell types by acting as a chemoattractant signal promoting recruitment of macrophages/phagocytes to the site of cells from which ICAM3 has been shed (described, for example, in Torr et al 2012, hereby incorporated by reference in its entirety). Cells recruited to the site of cells from which ICAM3 has been shed may include the immune cells that the present authors have shown are induced/mobilised by high concentrations of glucocorticoids, as described more fully below. The present authors hypothesize that ICAM3 shedding following dexamethasone binding may contribute to mobilisation of these novel immune cells after supra-high concentrations of glucocorticoids. ICAM3 shedding may also prevent the SARS-CoV-2 virus from entering cells via ICAM3 is a receptor for cell entry.

Thus, in some embodiments of the methods of the disclosure, the GR modulating agent causes ICAM3 shedding from the surface of a cell into the extracellular space. In some embodiments, at least about 10, 20, 30, 35, 40, 45 50, 55, 60, 65, 70, 75 80, 85, 90, 95, 96, 97, 98, 99, or 99% of the total ICAM3 expressed by a cell is shed into the extracellular space. In some embodiments, the ICAM3 elicits at least about a 10, 20, 30, 35, 40, 45, 50, 55, 60, 65, 70, 75 80, 85, 90, 95, 96, 97, 98, 99, or 99% reduction in surface expression of ICAM3 on a cell. In some preferred embodiments, at least about 30 or 40% of the total ICAM3 expressed by a cell is shed into the extracellular space. In some preferred embodiments, the ICAM3 elicits at least about a 35 or 40% reduction in surface expression of ICAM3 on a cell. Suitable methods for determining extent and changes in ICAM3 expression and shedding are well known to those of skill in the art, for example the methods described in Juan et al (which is hereby incorporated by reference in its entirety).

Biological Action Via Induction of Immune Cells

High doses of glucocorticoid exert powerful biological effects that will contribute to the treatment of COVID-19. AVM0703 at human-equivalent dose (HED doses) of 18 mg/kg and above mobilizes a very active Natural Killer T cell that expresses CD3 at very high levels (AVM_NKT cells), which are not otherwise found in the circulation without this high dose treatment. This cell type is discussed in greater detail below. Thus, glucocorticoid binding to ICAM3 may lead to the production and mobilization of novel Natural Killer T (NKT) cells from the spleen, thymus, or bone marrow into the circulation. These novel NKT cells can identify and target SAR CoV2 infected cells for destruction.

At HED of 18 mg/kg dexamethasone and higher, besides mobilizing the abovementioned AVM_NKT cells, novel CD3-very-high T cell, and CD11b-very-high dendritic cells are also induced. Surprisingly, these doses of glucocorticoid do not appear to activate GRs because they have no activity at equivalent concentrations in vitro on whole blood or splenocytes (see Example 2, below), and in vivo there have been no effects on colon, pancreas or bone, which would be expected if GRs were being activated. In vivo only lymphocytes, monocytes, some neutrophils and cancer cells have been ablated. Almost complete ablation is seen just 6 hours after dexamethasone dosing, demonstrating a rapid onset of action that may be preferable to slower acting COVID-19 alternatives, which can take days for effect. Most importantly, the T lymphocyte component of the novel NKT populations described herein may provide long term immunity for the patient that would be protective if they are exposed to the virus again.

As severe COVID-19 patients are reported to already have lymphopenia (study supported by Natural Science Foundation of Guangzhou; S2018010009732, author not disclosed; and Xu et al 2020) the inventors do not foresee any worsening effect on lymphocyte count caused by the high dose glucocorticoids according to the invention. Moreover, by mobilizing CD3-very-high NK, CD3-very-high T cells and CD11b-very-high dendritic cells, this treatment triggers a mechanism for directly killing and engulf the virus. The same study reporting lymphopenia did not see a decrease in the typical NKT cells in severe COVID-19 patients. AVM-NKT cells were not present in the blood of any COVID-19 patient, as no cells were observed expressing CD3 at 1-1.5 log higher mean fluorescent intensity (MFI) than the typical NKT, indicating that endogenous cortisol is not sufficient in COVID-19 patients to mobilize this AVM_NKT cell population.

AVM_NKT cells are NKp46+ and Ly6G positive, indicating the potential to directly kill targets as well as to engulf them. The typical AVM_NKT summarized above which are CD1d restricted do not express Ly6G. CD11b very high dendritic cells are also activated by these doses of AVM0703. In vivo, glucocorticoids ablates lymphocytes other than NK and NKT cells, and monocytes, within 6 hours after oral dosing of naïve mice. In tumor models the novel NKT and T cell are not observed in the blood. However, within 48 hours of dosing NKp46+ cells and Ly6G+ cells, which are not macrophages as they are negative for F4/80, can be found in formations within the tumors, surrounding regions of remaining viable tumor. This indicates the functional potency of these cells.

Beyond the known properties of NKT cells and the advantages of NKT cell-targeted immunotherapy, the novel AVM_NKT cells offer an additional advantage as they may add direct target cell engulfment to the known killing properties of other induced NKT (iNKT) cells. This function is attributed to the AVM_NKT's unique expression of Ly6G and TCRγδ. Moreover, the AVM_NKT are induced by high dose glucocorticoids and are thus, in principle, available in large numbers, in contrast to the limited numbers of natural NKT (and iNKT cells) that are insufficient for autologous therapy in the elderly, who are most vulnerable to COVID-19.

Glucocorticoid Receptor Modulating Agents

As used herein, the term glucocorticoid-receptor (GR) modulating agent includes glucocorticoids, glucocorticoid receptor agonists, and any compound that binds to the glucocorticoid receptor. Glucocorticoid-receptor (GR) modulating agents such as glucocorticoids exert their effects through both membrane GRs and cytoplasmic GRs which activate or repress gene expression. Some of the desirable lymphodepletive effects of glucocorticoids and GR modulating agents are believed to be mediated via membrane GRs or other non-genomic effects in addition to their genomic effects. Glucocorticoids have been reported to have varied effects on lymphocyte levels, depending on the concentration of the glucocorticoid administered and the duration of treatment. In general, at low doses typically used for chronic therapy, glucocorticoids have been reported to redistribute lymphocytes from the peripheral blood into the bone marrow, at medium doses glucocorticoids have been reported to cause leukocytosis thought to be a redistribution of leukocytes from the bone marrow, spleen and thymus into the peripheral blood, and at high doses glucocorticoids have a lymphotoxic action on lymphocytes by triggering apoptosis and necroptosis. The duration of effect also depends on the dose level; for instance Fauci et al (1976) reports a single oral 0.24 mg/kg dexamethasone dose suppresses peripheral blood T and B lymphocytes 80% with recovery beginning at 12 hours and normal levels by 24 hours. The present authors have previously demonstrated (in international patent application PCT/US2019/054395) that acute oral doses of 3 mg/kg or greater dexamethasone are necessary to reduce peripheral blood T and B cells 24-48 hours after administration, with return to baseline levels occurring around 5 to 14 days after dosing.

In some embodiments of the methods of the disclosure, the glucocorticoid-receptor (GR) modulating agent is a glucocorticoid. In some such embodiments, the glucocorticoid may be selected from the group consisting of: dexamethasone, hydrocortisone, methylprednisolone, prednisone, prednisolone, prednylidene, cortisone, budesonide, betamethasone, flumethasone and beclomethasone. In some preferred embodiments, the glucocorticoid may be selected from the group consisting of: dexamethasone, betamethasone, and methylprednisone. In some particularly preferred embodiments the glucocorticoid may be dexamethasone or betamethasone.

In some embodiments of the methods of the disclosure, the glucocorticoid may be dexamethasone, e.g. dexamethasone base, dexamethasone sodium phosphate, dexamethasone hemisuccinate, dexamethasone sodium succinate, dexamethasone succinate, dexamethasone isonicotinate, dexamethasone-21-acetate, dexamethasone phosphate, dexamethasone-21-phosphate, dexamethasone tebutate, dexamethasone-17-valerate, dexamethasone acetate monohydrate, dexamethasone pivalate, dexamethasone palmitate, dexamethasone-21-palmitate, dexamethasone dipropionate, dexamethasone propionate, dexamethasone acetate anhydrous, dexamethasone-21-phenylpropionate, dexamethasone-21-sulfobenzoate, dexamethasone hemo-sulfate, dexamethasone sulfate, dexamethasone beloxil, dexamethasone acid, dexamethasone acefurate, dexamethasone carboximide, dexamethasone cipecilate, dexamethasone 21-phosphate disodium salt, dexamethasone mesylate, dexamethasone linoleate, dexamethasone glucoside, dexamethasone glucuronide, dexamethasone iodoacetate, dexamethasone oxetanone, carboxymethylthio-dexamethasone, dexamethasonebisethoximes, dexamethasone epoxide, dexamethasonelinolelaidate, dexamethasone methylorthovalerate, dexamethasone spermine, 6-hydroxy dexamethasone, dexamethasone tributylacetate, dexamethasone aspartic acid, dexamethasone galactopyranose, dexamethasone hydrochloride, hydroxy dexamethasone, carboxy dexamethasone, desoxy dexamethasone, dexamethasone butazone, dexamethasone cyclodextrin, dihydro dexamethasone, oxo dexamethasone, propionyloxy dexamethasone, dexamethasone galactodie, dexamethasone isonicotinate, dexamethasone sodium hydrogen phosphate, dexamethasone aldehyde, dexamethasone pivlate, dexamethasone tridecylate, dexamethasone crotonate, dexamethasone methanesulfonate, dexamethasone butylacetate, dehydro dexamethasone, dexamethasone Isothiocyanatoethyl)Thioether, dexamethasone bromoacetate, dexamethasone hemiglutarate, deoxy dexamethasone, dexamethasone chlorambucilate, dexamethasone melphalanate, formyloxy dexamethasone, dexamethasone butyrate, dexamethasone laurate, dexamethasone acetate, and any combination treatment that contains a form of dexamethasone. In some preferred embodiments, the glucocorticoid may be dexamethasone base or dexamethasone sodium phosphate.

Glucocorticoid-receptor (GR) modulating agents which may be used in the disclosed methods include, for example, selective glucocorticoid receptor modulators (SEGRMs) and selective glucocorticoid receptor agonists (SEGRAs). Glucocorticoids, selective glucocorticoid receptor modulators, and selective glucocorticoid receptor agonists (SEGRAs) that may be utilized in the disclosed methods are well known to those skilled in the art.

Some such glucocorticoids include, but are not limited to, dexamethasone, dexamethasone containing agents, hydrocortisone, methylpredisone, prednisone, corticone, budesonide, betamethasone and beclomethasone. Other glucocorticoids include prednisolone, mometasone furoate, Triamcinolone Acetonide, and methylprednisolone.

In some embodiments, the glucocorticoid receptor modulating agent may not be one or more of the above recited agents.

High Dose Glucocorticoid Formulations

The inventors have previously shown that stable high-concentration glucocorticoid formulations can be prepared even when the formulation comprises reduced or no amounts of preservative (e.g. antioxidants). One aspect of the high-concentration glucocorticoid formulations that ensures good stability is the ratio of headspace volume (ml) to glucocorticoid (mg). For instance, the inventors have shown, in international patent application PCT/US2019/061363, that a Dexamethasone Sodium Phosphate solution called AVM0703 is particularly favored. AVM0703 contains 26.23 mg/mL dexamethasone sodium phosphate (equivalent to 24 mg/mL dexamethasone phosphate, DP), 10 mg/mL sodium citrate, 0.5 mg/mL disodium edetate, and 0.035 mg/mL sodium sulfite (anhydrous). These high concentration glucocorticoid formulations find utility in some embodiments of the methods and treatments of the present invention.

Glucocorticoid Dosage Calculations

In the methods of the disclosure, the glucocorticoid-receptor (GR) modulating agent is administered at a high dose, preferably at a dose equivalent to about at least 18 mg/kg human equivalent dose (HED) of dexamethasone base. Equivalent doses of another glucocorticoid or glucocorticoid receptor modulating agent can be readily and easily calculated using publicly available corticoid conversion algorithms, preferably http://www.medcalc.com. By way of example, 18 mg/kg dexamethasone converts to 112.5 mg/kg prednisone. Since prednisone's biologic half-life is about 20 hours, while dexamethasone's biologic half-life is about 36 to 54 hours, prednisone would be dosed at about 112.5 mg/kg every 24 hours for equivalent biologic dosing. More specifically, an 18 mg/kg dose of dexamethasone corresponds to a 112.5 mg/kg dose of prednisolone that would require repeat dosing of about two to about three doses every 24 hours. A 10 mg/kg dose of betamethasone is about 12 mg/kg dexamethasone and has a pharmacodynamic (biologic) half-life similar to dexamethasone.

Dexamethasone doses in the present application are given as human equivalent doses (HED). Methods for calculating the human equivalent dose (HED) are known in the art. For example the FDA's Centre for Drug Evaluation and Research (CDER) issued a highly-cited guidance document in 2005 (U.S. Department of Health CDER, 2005), which sets out the established algorithm for converting animal doses to HED based on body surface area (the generally accepted method for extrapolating doses between species) at Table 1 on page 7 of that document. For reference, Table 1 is reproduced below. The skilled person understands that the animal dose in mg/kg, explained below, the HED is calculated easily using the standard conversion factors in the right hand columns of Table 1:

TABLE 1
Conversion of Animal Doses to Human Equivalent
Doses Based on Body Surface Area

| | | To Convert Animal Dose in | |
|---|---|---|---|
| | | mg/kg to HED^{a} in mg/kg, | |
| | To Convert Animal | Either: | |
| | Dose in mg/kg to | Divide | Multiply |
| | Dose in mg/m², | Animal Dose | Animal Dose |
| Species | Multiply by kₘ | By | By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |

Primates:

| | | | |
|---|---|---|---|
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, rhesus, and stumptail.

In some embodiments of the methods of the disclosure, the glucocorticoid-receptor (GR) modulating agent may be administered at a dose equivalent to about at least 6 mg/kg, 12 mg/kg, 15 mg/kg, 18 mg/kg, 24 mg/kg, 30 mg/kg, or about at least 45 mg/kg human equivalent dose (HED) of dexamethasone base. In some preferred embodiments, the glucocorticoid-receptor (GR) modulating agent may be administered at a dose equivalent to about 6 mg/kg, 12 mg/kg, 15 mg/kg, 18 mg/kg, 24 mg/kg, 30 mg/kg, or about 45 mg/kg human equivalent dose (HED) of dexamethasone base, or at a dose that is at least a dosage value that is equivalent to about 6 mg/kg, about 12 mg/kg, about 15 mg/kg, about 18 mg/kg, about 24 mg/kg, about 30 mg/kg, or about 45 mg/kg HED of dexamethasone base. In some preferred embodiments, the glucocorticoid-receptor (GR) modulating agent may be administered at a dose equivalent to about at least 6 mg/kg human equivalent dose (HED) of dexamethasone base. In some other preferred embodiments, the glucocorticoid-receptor (GR) modulating agent may be administered at a dose equivalent to about at least 18 mg/kg human equivalent dose (HED) of dexamethasone base.

In some embodiments of the methods of the disclosure, the glucocorticoid-receptor (GR) modulating agent may be administered at a dose equivalent to about at least 6-45 mg/kg human equivalent dose (HED) of dexamethasone base; about at least 15-24 mg/kg human equivalent dose (HED) of dexamethasone base; about at least 6-12 mg/kg human equivalent dose (HED) of dexamethasone base; or about at least 12-15 mg/kg human equivalent dose (HED) of dexamethasone base; or about at least 18-30 mg/kg human equivalent dose (HED) of dexamethasone base.

In the methods of the disclosure, the glucocorticoid-receptor (GR) modulating agent may be administered as a single acute dose, or as a total dose given over about a 24, 48, or 72 hour period. In some preferred embodiments, the glucocorticoid-receptor (GR) modulating agent is administered as a single acute dose. In other preferred embodiments, the glucocorticoid-receptor (GR) modulating agent is administered as a total dose given over about a 72 hour period.

Administration Routes

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically.

The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others.

The term ‘site of injection’ as used herein non-exclusively relates to intra-tumor, or intra-organ such as the kidney or liver or pancreas or heart or lung or brain or spleen or eye, intra-muscular, intro-ocular, intra-striatal, intradermal, by dermal patch, by skin patch, by patch, into the cerebrospinal fluid, into the brain, among others. In some preferred embodiments of the disclosure, the glucocorticoid-receptor modulating agents may be administered orally.

In some embodiments of the disclosure, the route of administration for the glucocorticoid-receptor modulating agents disclosed herein may not be one or more of the above recited routes.

Pharmaceutical Compositions

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. glucocorticoids, used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

Cell Types

Dendritic cells (DCs) are bone marrow-derived leukocytes, and are the most potent antigen-presenting cells of the mammalian immune system. DCs exert immune-surveillance for exogenous and endogenous antigens and the later activation of naive T lymphocytes giving rise to various immunological responses. DCs are sentinel cells responsible for the recognition of pathogens and signals of tissue damage, which induces their migration to lymphoid organs to carry out the activation of different subsets of T, natural killer (NK), NKT, and B lymphocytes. Mature phenotype cDC are characterized by an increase in MHCII, CD80, CD86, and CD40. Dendritic cells are frequently classified into conventional dendritic cell (cDC) and plasmacytoid dendritic cell (pDC) subsets. Dendritic cells exist primarily in two basic functional states: “immature” and “mature”. Activation (maturation) of dendritic cells turns on metabolic, cellular, and gene transcription programs allowing DC to migrate from peripheral tissues to T-dependent areas in secondary lymphoid organs, where T lymphocyte-activating antigen presentation may occur (Patente et al, 2018; hereby incorporated by reference in its entirety). The main function of dendritic cells is to process antigen material and present it on the cell surface to T cells thus initiating adaptive immune responses. Dendritic cells also produce polarizing cytokines that promote pathogen-specific effector T cell differentiation and activation, and can promote self-tolerance by secreting tolerogenic cytokines that induce the differentiation of regulatory T cells. The DCs induced by the methods of the invention may express CD11b to a very high level (“CD11b-very-high dendritic cells”).

T cells are a type of lymphocyte that play a key role in the immune response. T cells are distinguished from other types of lymphocytes by the presence of T-cell receptors on their cell surface. T-cell receptors (TCRs) are responsible for recognizing fragments of antigen bound to major histocompatibility complex (MHC) molecules, and are heterodimers of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes in diseased states (such as leukemia). In contrast to MHC-restricted alpha beta T cells, gamma delta T cells do not require antigen processing and major-histocompatibility-complex (MHC) presentation of peptide epitopes for activation, although some recognize MHC class Ib molecules. Some gamma delta T cells recognise markers of cellular stress resulting from infection or tumorigenesis. Gamma delta T cells are also believed to have a role in recognition of lipid antigens. The T cells induced by the methods of the invention may express CD3 to a very high level (“CD3-very-high”).

Natural Killer T Cells (NKTs) are a heterogeneous group of T cells that share properties of both T cells and natural killer (NK) cells. In contrast to conventional T cells, NKTs are functionally mature when they exit the thymus, primed for rapid cytokine production. NKTs can directly kill CD1d expressing cancer cells and tumor microenvironment macrophages, rapidly produce and release immune activating cytokines such as IFNgamma and IL-4, and activate other immune cells such as dendritic cells (DCs), NK cells, and B and T lymphocytes.

The inventors have found a novel type of NKT cells, denoted AVM_NKT, which homes remarkably well to diseased tissues such as tumors in tumor killing models used to show efficacy of checkpoint inhibitors. AVM_NKT are mobilized only after AVM0703 treatment, as opposed to other NK and NKT which circulate continuously, therefore AVM_NKT numbers are unlimited in practice. These cells have been shown to reduce Influenza A mediated inflammation and disease severity, and CD11b+ DC have been implicated in protection against Respiratory Syncytial Virus and Influenza A (H1N1). Therefore, cells with lower Cd3 and CD11b levels are known to be effective, AVM0703 should be more effective because of the CD3 very high NKT and gamma/delta T cells it mobilizes, and because it not only increases the number of conventional CD11b DCs, it also mobilizes a CD11b very high expressing DC not typically observed.

Alveolar type II (AT2) cells are thought to be a target for SARS-Cov-2 in the human lung. A2T cells are the only pulmonary cells to synthesize, store and secretes all components of pulmonary surfactant important for surface tension regulation, atelectasis prevention and maintaining alveolar fluid balance within the alveolus (Henry et al, 2017).

Determining Cell Marker Expression Levels

Markers expressed by NKT cells, T cells or DCs induced by the methods of the invention can be determined by reference to the level of marker expression by a typical NKT cell, T cell or DC population (respectively), e.g. taken from the patient prior to treatment. Where the expression level is said to be “very high” this can denote 50%, 60%, 70%, 80%, 90% or 100% higher level expression of the marker compared to the respective typical cell population. Expression levels may be determined by flow cytometry.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example I—Sequence Alignment of Human DC-SIGN, L-SIGN, SARS-Cov and SARS-Cov-2 Spike Glycoproteins

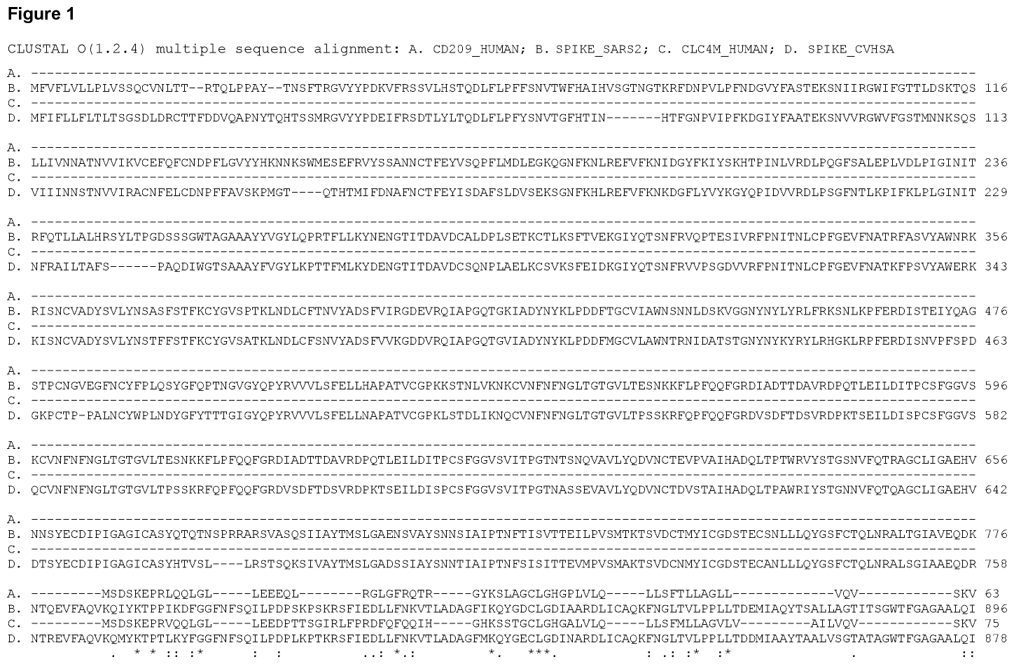

Clustal 0(1.2.4) multiple sequence alignment tool (uniprot.org) was used to align the gene products of human 00209 (DC-SIGN), CLEC4M (L-SIGN) with the spike glycoproteins of SARS-CoV and SARS-Cov-2. The results are shown in FIG. 1. The spike glycoproteins show clear homology to the ICAM3-binding regions of DC-SIGN and L-SIGN.

### Example 2—Absent Ex Vivo Effect of High Concentration Dexamethasone on Whole Blood or Splenocytes

Ex vivo whole blood or isolated splenocytes had no response to AVM73 or dexamethasone base at concentrations between 100 uM up to 500 uM, the equivalent concentrations attained at peak in vivo dosing around Human Equivalent Dose (HED) 6 mg/kg and higher. These data demonstrate that at the doses preferred for cancer and infectious disease treatments (HED 6 mg/kg up to 30 mg/kg) AVM0703 does not activate glucocorticoid receptors nor a receptor directly expressed on lymphocytes, monocytes or neutrophils. The profound in vivo activity that we observe in naïve mice and in cancer models must be mediated through the mobilized supercharged Natural Killer T cells, T cells and dendritic cells after AVM0703 HED 6 mg/kg and higher.

The incubation conditions shown in Table 2 were investigated.

TABLE 2
Incubation conditions

| | | Volume | | Temperature | CO₂ | | Timepoint |
|---|---|---|---|---|---|---|---|
| Study No. | Cell Type | (μL) | Container | (° C.) | (%) | Agitation | (hr) |
| 6 | Whole | 300 | 1.5 mL | Room | Atmospheric | None | 16 |
| | Blood | | microcentrifuge | temperature | | | |
| | | | tube | | | | |
| 24 | Whole | 400 | 24-well plate | Room | Atmospheric | Orbital | 16 |
| | Blood | | | temperature | | | |
| | Splenocytes | 500 | 24-well plate | 37 | 3% | None | 4 |
| Venetoclax 1 | Whole | See | 1.5 mL | Room | Atmospheric | None | 2, 22 |
| | blood | Table 2 | microcentrifuge | temperature | | | |
| | | | tube | | | | |
| Glucocorticoid | Whole | See | 1.5 mL | Room | Atmospheric | None | Overnight |
| Comparison | blood | Table 4 | microcentrifuge | temperature | | | |
| | | | tube | | | | |

Results; Whole Blood (Study 6)

Samples were treated in triplicate with either 500 μM dexamethasone base, 50 μM RU486 (a steroid receptor antagonist), or 1% DMSO as control. A non-significant increase in neutrophils induced by RU486 treatment. Other immune cells types; lymphocytes, monocytes, eosinophils, basophils; displayed no change in numbers with dexamethasone base or RU486 treatment. The data indicates that dexamethasone base alone in whole blood does not induce the lymphocyte-killing activity seen in in vivo experiments. RU486 alone also has no killing activity on lymphocytes and can be given in combination without impacting lymphocyte levels.

Results; Splenocytes (Study 24)

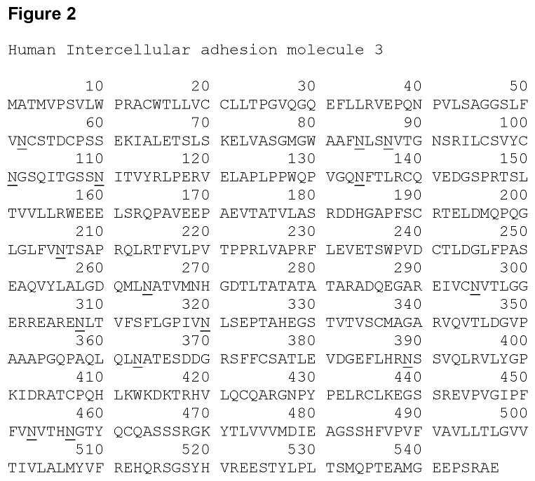

Splenocytes were cultured for 16 hours prior to assay execution. Flow cytometry was performed at four after dexamethasone base addition, and the results are shown in FIG. 3. After 4 hours, dexamethasone base appears to have direct killing activity at concentrations lower than 100 μM, but does not trigger cell death above 100 μM.

The results of Study 6 and 24 suggest that the mechanism of action is different between low and high concentrations of dexamethasone base, where high doses have no activity on splenocytes. This is in contrast to previous in vivo data, where the weight of the spleen is sharply reduced by AVM0703 dosing. (See WO2018/183927 and WO2020/072713; both incorporated by reference in their entirety, for discussion of these in vivo effects.) The discrepancy between ex vivo and in vivo indicates that a different mechanism of action is occurring in vivo compared to ex vivo.

Results; Venetoclax 1 No significant difference was found for any measured parameter of CBC when whole blood was incubated with Placebo, DMSO, AVM0703 at 500 μM alone, or combined with venetoclax at 3 μM. Particularly, there is no significant effect on white blood cells. Lymphocytes, monocytes, and neutrophils do not exhibit the lymphodepletion or ablation observed in vivo. Thus, these results indicate that cells in whole blood are not influenced by AVM0703 via GCR activation (venetoclax increases glucocorticoid sensitivity).

Results; Ex Vivo Glucocorticoid Comparison

To determine whether the absence of ex vivo effect on monocytes, lymphocytes, and neutrophils was due to the concentration of dexamethasone base of 100 uM to 500 uM, or whether the different excipients in AVM0703 could be contributing we compared ex vivo treatment followed by CBC analysis among AVM0703 compared with other commercially available Dexamethasone Phosphate. There were no noticeable differences between the different formulations, indicating that it is the concentrations of dexamethasone base that have an unexpected ex vivo effect; i.e. the surprising lack of ex vivo cell death.

### Example 3—Modelling the Binding Modes and Energies of SARS-CoV-2 and SARS-CoV Receptor Binding Domains with ICAM3 and ACE2

The inventors used ClusPro to look for favorable binding modes (models) between the receptor binding domain (RBD) of SARS-CoV-2 and ICAM3 and ACE2. The inventors then used Rosetta software to predict the energetics of each binding mode (model).

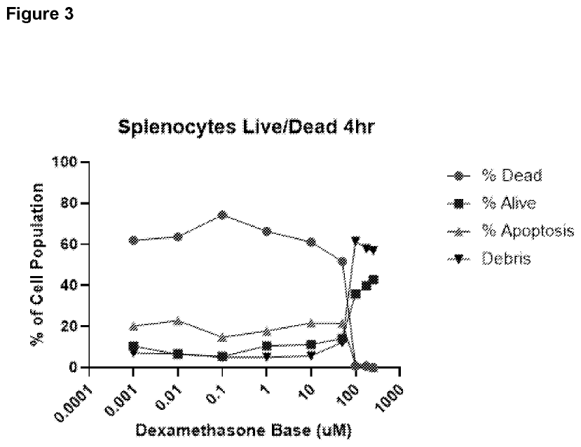

The left hand side of FIG. 4 shows an exemplary binding mode of the RBD of SARS-CoV-2 with ACE2. The graph on the right hand side of FIG. 4 plots the route mean squared deviance (RMSD) at the interface of the molecules against the Interface Score, which predicts binding energy (a more negative value denotes a more exothermic and thus stronger binding interaction). The “funneling down” of the Interface Scores towards the low extremity of the interface RMSD axis indicates favorable binding characteristics between the RBD of SARS-CoV-2 and ACE2.

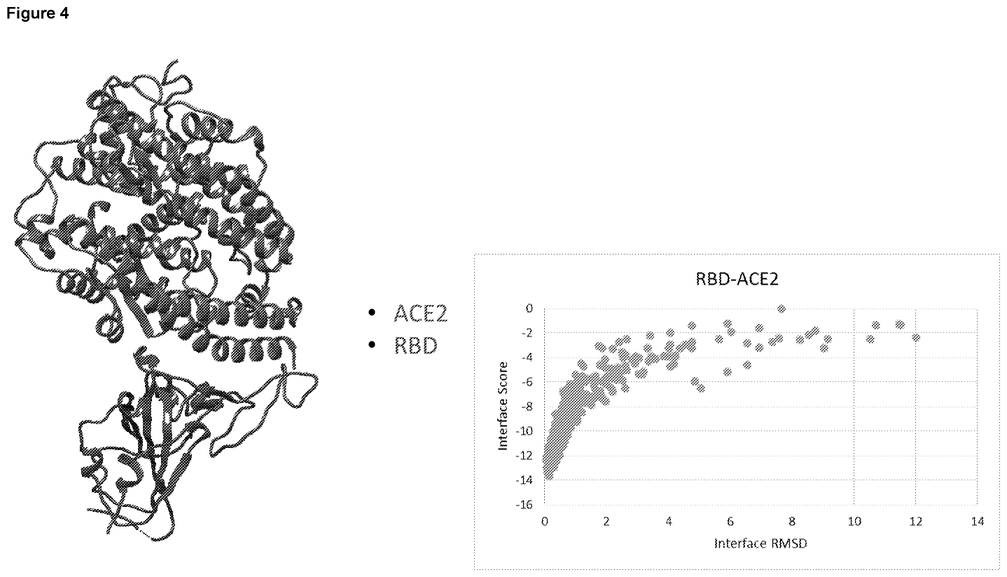

The left hand side of FIG. 5 shows an exemplary binding mode of the RBD of SARS-CoV-2 with ICAM3. ICAM3 docks in the RBD pocket of the CoV-2 SPIKE protein, as ACE2 does (unlike the binding mode of DC-SIGN or L-SIGN, which dock outside the CoV1 SPIKE RBD). The CoV-2 RBD-ICAM3 binding mode predicts a first Salt Bridge between Arg408 of the SARS-CoV-2 RBD and Glu 43 of ICAM3, and a second Salt Bridge between Arg6 and Glu484, as well as hydrophobic interactions in the central portion of the binding interface. The graph on the right hand side of FIG. 5 plots the (RMSD) at the interface of CoV-2 and ICAM3 against the Interface Score. As in FIG. 4, the “funneling down” of the Interface Scores towards the low extremity of the interface RMSD axis in FIG. 5 indicates favorable binding characteristics between the RBD of SARS-CoV-2 and ICAM3.

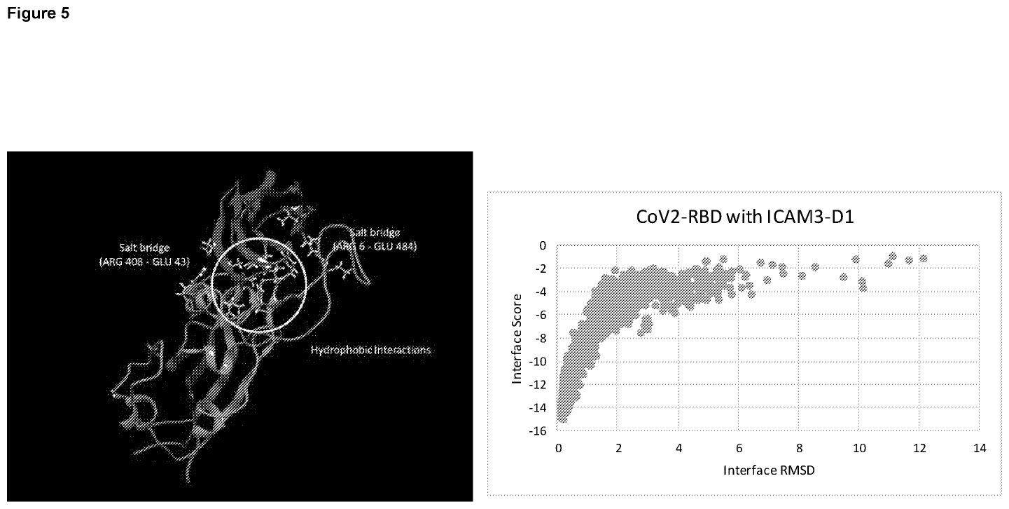

The left hand side of FIG. 6 shows an exemplary binding mode of the RBD of SARS-CoV-1 with ICAM3. The binding mode predicts a single Salt Bridge, between Arg395 of the RBD and Glu 43 of ICAM3. In contrast to FIGS. 4 and 5, the graph on the right hand side of FIG. 6 does not show a strong “funneling down” of the Interface Scores towards the low extremity of the interface RMSD axis. Thus, FIG. 6 indicating that the binding characteristics between the RBD of SARS-CoV-1 and ICAM3 are far less favorable than those between the RBD of SARS-CoV-2 and ICAM3 as indicated by FIG. 5.

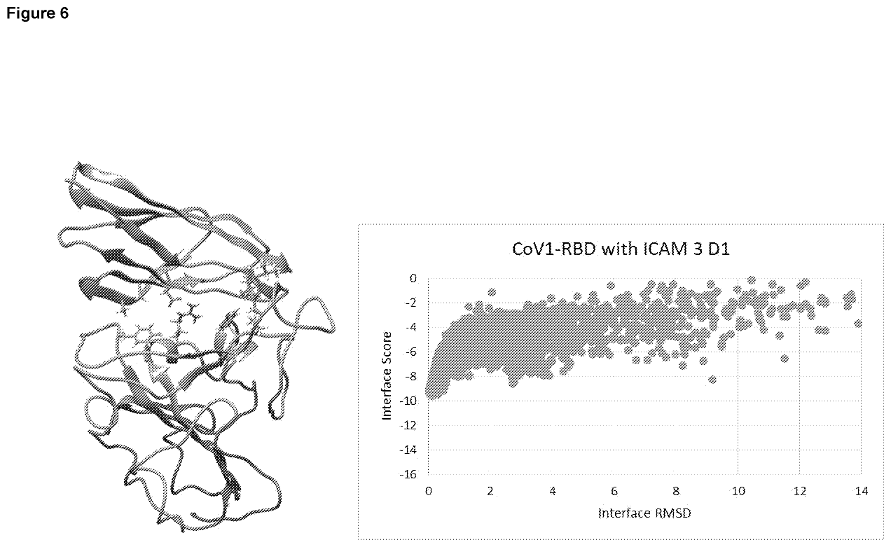

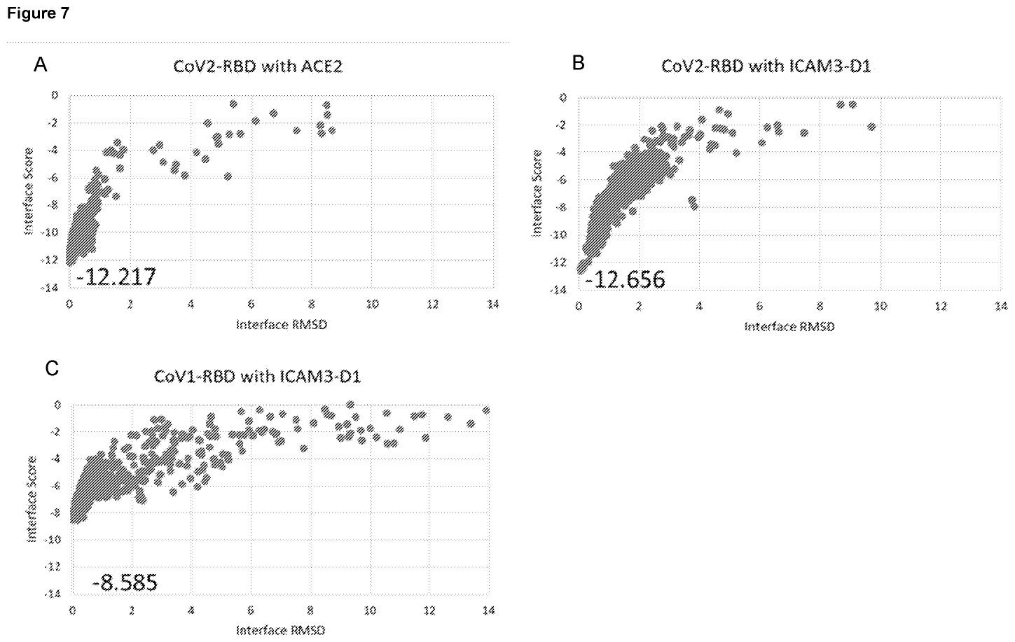

The results in FIGS. 4-6 were confirmed in an independent repeat of the modelling experiments using the Rosetta software to predict energetics of each binding mode. FIG. 7 shows the results of this series of repeat experiments—plotted are the route mean squared deviances (RMSD) at the interface of the molecules against the Interface Score, which predicts binding energy (a more negative value denotes a more exothermic and thus stronger binding interaction). As in FIGS. 4 and 5, a “funneling down” of the Interface Scores towards the low extremity of the interface RMSD axis indicates favorable binding characteristics between the RBD of SARS-CoV-2 and ACE2 (FIG. 7 A, corresponding to FIG. 4 right side) and between the RBD of SARS-CoV-2 and ICAM3 (FIG. 7 B, corresponding to FIG. 5 right side). Similarly, as in FIG. 6 no strong “funneling down” of the Interface Scores towards the low extremity of the interface RMSD axis was observed, indicating that the binding characteristics between the RBD of SARS-CoV-1 and ICAM3 are far less favorable than those between the RBD of SARS-CoV-2 and ICAM3 (FIG. 7C, corresponding to FIG. 6 right side). Rosetta energies for CoV2 RBD-ICAM3 (Interface RMSD −12.7) are as favorable as for ACE2 (−12.2) in contrast to CoV1-RBD-ICAM3 unfavorable energies (−8.6). ICAM3 docked in the CoV2 SPIKE RBD pocket (favorable electrostatic and hydrophobic interactions) as does ACE2, unlike DC- or L-SIGN which dock outside the CoV1 SPIKE RBD.

Preliminary MicroScale Thermophoresis experiments determined the CoV2 SPIKE-ICAM3 binding affinity (KD) to be between 5-10 nM, while that for CoV2 SPIKE-ACE2 was around 10 nM.

### Example 4—Modelling the Binding Interaction of the SARS-CoV-2 Receptor Binding Domain, ICAM3, and DC-SIGN

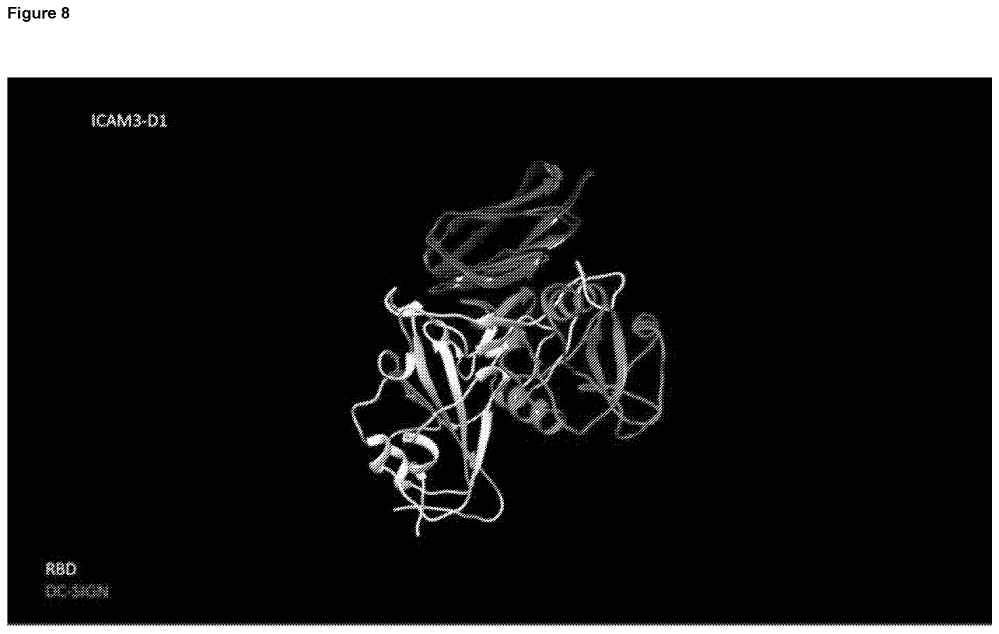

Modelling the binding interaction of the SARS-CoV-2 receptor binding domain, ICAM3, and DC-SIGN using ClusPro and Rosetta software, the inventors found that when the SARS CoV2 SPIKE RBD binds to ICAM3, then DC-SIGN cannot bind and vice versa (FIG. 8). This indicates that antibodies that are generated to the SARS CoV2 SPIKE RBD have the potential to cross-react and bind DC-SIGN, leading to unintended and undesired effects on the immune system.

### REFERENCES

A number of publications are cited above in order to more fully describe and disclose the invention and the state of the art to which the invention pertains. Full citations for these references are provided below. The entirety of each of these references is incorporated by reference herein.
Bhalla et al; Nat Commun 6, 6049 (2015)
Bhella; Philos Trans R Soc Lond B Biol Sci. (2015) February 5; 370; 1661
Barat et al, Journal of Virology May 2004, 78 (12) 6692-6697
Chan et al, The Journal of Infectious Diseases 2007; 196:271-80
Chan et al, Trends Microbiol, 21 (2013), pp. 544-555
Choi et al, Cytotherapy. (2017) October; 19(10):1248-1250
Fauci A. S., Pratt K. R. & Whalen G. (1976) J. Immunol. 118, 598.
Feng et al, Mol Cancer. 2020 Jun. 2; 19(1):100 Gregory & Pound, Apoptosis. 2010 September; 15(9):1029-49;
Juan et al, 1999, Allergy 54, 1293-1298
Han et al, 2007, Journal of Virology, Vol. 81, No. 21; November 2007 pp 12029-12039
Henry J. et al, Fetal and Neonatal Physiology (Fifth Edition), 2017
Horby et al, N Engl J Med. 2021 Feb. 25; 384(8):693-704
Hua et al, Front. Pharmacol., 4 Oct. 2013
Jeffers et al, Proc Natl Acad Sci USA. 2004 Nov. 2; 101(44):15748-53
Marzi et al, J Virol. 2004 November; 78(21):12090-5
Moffatt et al, J Immunol. 1999 Jun. 1; 162(11):6800-10;
Panesar, Med Hypotheses. 2008 August; 71(2):298-301
Patente et al; Front Immunol. 2018; 9: 3176
Qi, F. et al, Biochem. and Biophys. Res. Comm., https://doi.org/10.1016/j.bbrc.2020.03.044
Shen et al, Cancer letters vol. 422 (2018): 29-43. doi:10.1016/j.canlet.2018.02.034
Shi et al, 2020 DOI: 10.1101/2020.03.12.20034736
Siljan et al, 2018. DOI: 10.1093/ofid/ofy002
Sommerfelt, M. A., and B. Asjo (1995) J. Gen. Virol. 76:1345-1352
Song et al, 2005, PNAS; 102, 9, 3366-3371
Staunton et al (1989) Cell 56, 849-853
Torr et al, Cell Death Differ. 2012 April; 19(4): 671-679;
Wang et al, Int. J. of Antimicrobial Agents (2020), ANTAGE 105948
Wang et al, Cell Mol Immunol. 2020 Apr. 7: 1-3
Wrapp et al, Science, 13 Mar. 2020: 367, Issue 6483, pp. 1260-1263
Wu et al, Cell host & microbe (2020) 27, Issue 3, 11 Mar. 2020, Pages 325-328
Xu et al, 2020 Feb. 18 doi: 10.1016/S2213-2600(20)30076-X PMCID: PMC7164771 PMID: 32085846
Zhang et al, Front Immunol. 2020; 11: 2033

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method of inhibiting a SARS-Cov-2 virus from entering a host cell, the method comprising contacting the cell with a glucocorticoid receptor (GR) modulating agent.

2. The method of claim 1, wherein the GR modulating agent acts by binding to Intercellular Adhesion Molecule 3 (ICAM3) present at the surface of the host cell.

3. The method of claim 2, wherein the GR modulating agent acts as an ICAM3 antagonist.

4. The method of claim 3, wherein the GR modulating agent inhibits the spike (S) glycoprotein of SARS-Cov-2 from binding to the ICAM3 present at the surface of the host cell.

5. The method of any one of claims 1-4, wherein the GR modulating agent causes ICAM3 shedding from the surface of the host cell into the extracellular space.

6. The method of claim 1, wherein the GR modulating agent contacts an additional cell and causes ICAM3 shedding from the surface of the additional cell into the extracellular space.

7. The method of claim 5 or 6, wherein ICAM3 that has been shed into the extracellular space inhibits SARS-Cov-2 binding to the surface of the host cell.

8. The method of claim 7, wherein ICAM3 that has been shed into the extracellular space:
i) inhibits SARS-Cov-2 binding to L-SIGN and/or DC-SIGN at the surface of the host cell; and/or
ii) binds SARS-Cov-2, thereby reducing binding to ICAM3, L-SIGN and/or DC-SIGN at the surface of the host cell.

9. The method of claim 4, wherein the host cell is an immune cell.

10. The method of claim 9, wherein the immune cell is a lymphocyte, a monocyte, an eosinophil, a neutrophil or a dendritic cell.

11. The method of claim 8, wherein the host cell is a lung cell.

12. The method of claim 11, wherein the lung cell is an alveolar type-2 cell or a bronchiole cell.

13. The method of claim 1, wherein the GR modulating agent acts by binding SARS-Cov-2 to block its binding to its viral entry receptor on the host cell.

14. The method of any one of the preceding claims, wherein the GR modulating agent is a glucocorticoid.

15. The method of claim 14, wherein the glucocorticoid is selected from the group consisting of: dexamethasone, hydrocortisone, methylprednisolone, prednisone, prednisolone, prednylidene, cortisone, budesonide, betamethasone, flumethasone and beclomethasone.

16. The method of claim 15, wherein the glucocorticoid is dexamethasone.

17. A method of treating COVID-19 in a patient, the method comprising administering a glucocorticoid receptor (GR) modulating agent at a sufficiently high dose to inhibit SARS-Cov-2 particles from infecting a cell of the patient and/or to trigger or support an effective immune response to SARS-Cov-2 in the patient.

18. The method according to claim 17, wherein SARS-Cov-2 particles are inhibited from infecting an immune cell of the patient by binding to Intercellular Adhesion Molecule 3 (ICAM3) present at the surface of the immune cell.

19. The method according to claim 18, wherein the GR modulating agent acts as an ICAM3 antagonist.

20. The method according to claim 19, wherein the GR modulating agent inhibits the spike (S) glycoprotein of SARS-Cov-2 from binding to the ICAM3 present at the surface of the immune cell.

21. The method according to claim 20, wherein the immune cell is a lymphocyte, a monocyte, an eosinophil, a neutrophil or a dendritic cell.

22. The method according to claim 17, wherein SARS-Cov-2 particles are inhibited from infecting a lung cell of the patient, wherein the high dose GR modulating agent causes ICAM3 shedding into the extracellular space and wherein the ICAM3 that has been shed into the extracellular space inhibits SARS-Cov-2 binding to the surface of the lung cell.

23. The method according to claim 22, wherein ICAM3 that has been shed into the extracellular space inhibits SARS-Cov-2 binding to L-SIGN and/or DC-SIGN at the surface of the lung cell.

24. The method according to claim 23, wherein the lung cell is an alveolar type-2 cell or a bronchiole cell.

25. The method according to claim 17, wherein the GR modulating agent acts by binding a SARS-Cov-2 particle to block binding to its viral entry receptor on the cell of the patient.

26. The method according to claim 17, wherein the effective immune response involves the induction and/or mobilisation of a population of NKT cells that are characterized in that they expresses CD3, and:
a. express CD4, CD8, CD45, CD49b (CD56 in humans), CD62L, NK1.1, Ly6G, Sca1,
and/or TCR gamma/delta; and/or
b. do not express: C-kit, B220, FoxP3, and/or TCR alpha/beta.

27. The method according to claim 17, wherein the effective immune response involves the induction and/or mobilisation of a population of T cells that express CD3 to a very high level (“CD3-very-high”).

28. The method according to claim 17, wherein the effective immune response involves the induction and/or mobilisation of a population of dendritic cells (DCs) that express CD11b to a very high level (“CD11b-very-high dendritic cells”).

29. The method according to any one of claims 17-28, wherein the dose of the GR modulating agent is at least about 12 mg/kg, at least about 15 mg/kg, at least about 18 mg/kg, at least about 24 mg/kg, at least about 30 mg/kg, or at least about 45 mg/kg human equivalent dose (HED) of dexamethasone base.

30. The method according to any one of claims 17-29, wherein the GR modulating agent is a glucocorticoid, e.g. dexamethasone.
